(19) United States
(12) Patent Application Publication
(10) Pub. No.: US 2022/0339430 A1
(43) Pub. Date: Oct. 27, 2022

(54) UNIQUE THERAPEUTIC DOSE FOR NMES SYSTEMS

(71) Applicant: CyMedica Orthopedics, Inc., Scottsdale, AZ (US)

(72) Inventors: Kereshmeh Shahriari, Cave Creek, AZ (US); Struan Coleman, Locust Valley, NY (US)

(21) Appl. No.: 17/727,502

(22) Filed: Apr. 22, 2022

Related U.S. Application Data
(60) Provisional application No. 63/178,909, filed on Apr. 23, 2021.

(51) Int. Cl. A61N 1/04; A61N 1/36

(52) U.S. Cl. CPC A61N 1/0452; A61N 1/0476; A61N 1/0484; A61N 1/36034

(57) ABSTRACT

This disclosure describes various aspects of a unique therapeutic does for an NMES system. In some embodiments, the system includes one or more electrodes and one or more controls that execute a therapy session program. The therapy session program causes the controller to implement a pulse to the one or more electrodes that, when viewed on a graph of amplitude vs. time, create a “chair” shape with an exponentially decreasing amplitude. In some embodiments, the pulse is considerably longer (e.g., 5 ms) than those implemented in the past (e.g., 300 μs). In some embodiments, the pulse is held at a non-zero amplitude after an exponential decrease for a period of time before a non-electrical relaxation time period is implemented by the one or more controllers.

### CROSS-REFERENCE TO RELATED APPLICATIONS

This application claims the benefit of U.S. Provisional Patent Application No. 63/178,809, filed Apr. 23, 2021, entitled, “UNIQUE THERAPEUTIC DOSE FOR NMES SYSTEMS,” the entire contents of which are incorporated herein by reference. This application and the unique therapeutic dose disclosed herein is combinable with, implementable, and/or enabled by the systems, structures, programs, and methods described in commonly owned U.S. Pat. No. 10,315,032 B2 which is incorporated by reference in its entirety.

### BACKGROUND

After an injury, it is important to have a monitored rehabilitation process to assist with healing and regaining full range of motion. Neuromuscular Electrical Stimulation (NMES) is sometimes used as part of the rehabilitation process to help a patient regain the ability to voluntarily contract their muscles. Currently, NMES application profiles attempt to mimic neurological signals to cause a targeted muscle to contract. These electrical pulse profiles often take the form of a square wave or sinusoidal profile that simply applies an on/off duty cycle. Little consideration has been given to the shape of the electrical pulse profile in the prior art.

Therefore, there is a need to create novel NMES devices to deliver one or more unique electrical pulse profiles implementing a unique therapeutic dose which results in greater pain alleviation and promotes better healing.

### SUMMARY

Some embodiments described herein are direct to a unique therapeutic system that creates a unique NMES electrical pulse profile implemented as a unique therapeutic dose. In some embodiments, a system for providing a unique neuromuscular electrical stimulation (NMES) therapeutic dose comprises one or more electrodes and/or one or more controllers. In some embodiments, the at one or more electrodes are coupled to the one or more controllers. In some embodiments, the one or more controllers are configured to control an electrical stimulation pulse supplied from an electrical source to the one or more electrodes. In some embodiments, the electrical stimulation pulse is not sinusoidal. In some embodiments, the electrical stimulation pulse is not a square wave. In some embodiments, the electrical stimulation pulse comprises one or more linear profiles and one or more non-linear profiles.

In some embodiments, a duration of the electrical stimulation pulse is greater than 1 ms. In some embodiments, a duration of the electrical stimulation pulse is between 3 and 7 ms. In some embodiments, a duration of the electrical stimulation pulse is between 4 and 6 ms. In some embodiments, a duration of the electrical stimulation pulse is substantially 5 ms. In some embodiments, the therapy session program is configured to execute for 17 to 23 minutes. In some embodiments, the therapy session program is configured to execute for 19 to 21 minutes. In some embodiments, the therapy session program is configured to execute for substantially 20 minutes. In some embodiments, the therapy session program comprises between 8 and 15 minutes of muscle contraction.

In some embodiments, the one or more controllers are configured to enable a user to adjust an intensity of the electrical stimulation pulse. In some embodiments, the electrical stimulation pulse is sufficient to cause muscle contraction when the electrode is placed on a user's skin. In some embodiments, the one or more controllers are configured to execute one or more therapy session programs. In some embodiments, the one or more therapy session programs comprises a plurality of cycles. In some embodiments, the plurality of cycles comprise one or more electrical stimulation time periods and one or more non-electrical relaxation time periods. In some embodiments, the one or more electrical stimulation time periods are configured to cause muscle contraction when the one or more electrodes are coupled to a user's skin. In some embodiments, the one or more non-electrical relaxation time periods are configured to not cause muscle contraction when the one or more electrodes are coupled to a user's skin.

In some embodiments, the one or more electrical stimulation time periods each comprise an electrical stimulation pulse comprising a pulse profile. In some embodiments, the pulse profile is one or more of asymmetrical, monophasic, and complex. In some embodiments, an electrical stimulation pulse between two non-electrical relaxation time periods comprises a pulse profile comprising an initial substantially linear increasing portion. In some embodiments, an electrical stimulation pulse between two non-electrical relaxation time periods comprises a pulse profile comprising a substantially exponentially decreasing pulse portion following a peak of the initial substantially linear increasing pulse portion. In some embodiments, the end of the substantially exponentially decreasing pulse portion comprises a substantially horizontal pulse section.

In some embodiments, the pulse profile comprises a substantially decreasing linear portion at an end of the substantially horizontal pulse section. In some embodiments, an end of the substantially decreasing linear portion joins with a non-electrical relaxation time period profile. In some embodiments, the substantially exponentially decreasing pulse profile does not exponentially decrease to a zero electrical power and/or does not exponentially decrease such that the substantially horizontal pulse section of the substantially exponentially decreasing pulse portion does not comprise a substantially horizontal transition into a non-electrical relaxation time profile.

In some embodiments, the exponentially decreasing profile comprises a substantially vertically decreasing section at the beginning of the decreasing profile and a substantially horizontal section at the end of the decreasing profile. In some embodiments, the pulse profile is defined on a graph of intensity and/or amplitude vs time, where the scale is such that the amplitude peak of the profile is at least twice the time width. In some embodiments, the controller is configured to maintain a non-zero electrical intensity execution at the substantially horizontal section for a predetermined time before a zero electrical intensity execution is implemented by the one or more controllers.

In some embodiments, a final decreasing profile following the exponentially decreasing profile comprises the zero electrical intensity execution. In some embodiments, the final decreasing profile comprises a substantially linear decreasing profile which joins to a non-electrical relaxation time period. In some embodiments, the final decreasing profile comprises a substantially vertically linear decreasing profile which joins to a non-electrical relaxation time period when the scale is such that the amplitude peak of the profile along a vertical amplitude axis is at least twice a width of the applied time along a horizontal time axis.

In some embodiments, a unique therapeutic dose includes an individual contraction time length per pulse. In some embodiments, a unique therapeutic dose includes a total contraction time length, where total contraction length is defined by work cycle duration multiplied by a number of muscle contractions. In some embodiments, a unique therapeutic dose includes a total net muscle contraction time, where total net muscle contraction time has been determined empirically to be approximately 11 minutes or 22 minutes per single session, where a session is defined as the application of an NMES waveform in a predetermined pattern for a predetermined period of time. In some embodiments, the controller is configured to implement muscle contraction according to the profile described herein for at least half of the execution time of the therapy execution program. In some embodiments, the therapy execution program includes a time duration between 20 and 40 minutes. In some embodiments, an NMES waveform and/or NMES waveform pattern includes one or more of pulse shape, treatment duration, frequency, pulse width, duty cycle, work cycle, relaxation time, contraction time, and rest time. In some embodiments, a cycle includes a single pulse in the NMES waveform.

In some embodiments, the one or more controllers are configured to control a total amount of time a unique therapeutic dose is applied per session. In some embodiments, the one or more controllers are configured to control a number of times the unique therapeutic dose is applied per day. In some embodiments, the one or more controllers are configured to control a number of times the unique therapeutic dose is applied per week. In some embodiments, the one or more controllers are configured to control a number of weeks the unique therapeutic dose is applied.

Some embodiments of the invention include a system comprising at least one sensor comprising a plurality of electrodes including at least one active electrode and at least one receiving electrode, the at least one sensor configured and arranged to be in physical contact with skin of a patient forming an electrical circuit with control electronics of at least one controller. The electrical circuit is configured and arranged to measure an electrical parameter using the at least one active electrode and at least one receiving electrode, and to form a closed loop electrical muscle stimulation system, where a stimulation current or voltage applied by the sensor onto the skin between the at least one active electrode and at least one receiving electrode is based on at least one program and at least one electrical parameter measured through the at least one active electrode and at least one receiving electrode.

Some embodiments include a computing program, applet or application configured to upload usage data for analysis. In some embodiments, at least one controller is configured and arranged to electromagnetically couple with a mobile computing device using at least a portion of the computing program, applet or application. In some embodiments, at least a portion of the computing program, applet or application is configured and arranged to include at least one user interface on a user's computing device, and the at least one user interface configured to display at least some usage data and to enable control of a parameter of the garment.

In some embodiments, at least one controller is configured to update the at least one user interface with at least one of a status of at least a portion of the garment, a position of at least a portion of the garment, and/or data from the at least one sensor.

In some embodiments, at least one user interface comprises a display including an option to scan and synchronize the controller with the at least one computer. Some embodiments include at least one user interface comprising a display including an option to scan and synchronize data stored on one or more non-transitory computer readable media. In some further embodiments, the at least one user interface comprises a display including an option to activate a wired or wireless link to connect a computer with the at least one controller. In other embodiments, the display is configured and arranged to enable the user to set or reconfigure the at least one stimulation pulse duration and/or intensity (amplitude).

Some embodiments include a display configurable by the at least a portion of the computing program, applet or application to display one or more parameters related to at least one of stimulation provided by at least a portion of the garment, and a range of motion measured by at least one sensor. In some embodiments, the display is configurable by the at least a portion of the computing program, applet or application to provide a visual representation of an action of a user wearing at least a portion of the garment. In some embodiments, the garment comprises a brace assembly. In some embodiments, the brace assembly comprises at least one of a brace, a stay, a sleeve, a band, a sling, a garment, a wrap, and a strap.

In some embodiments, at least one sensor comprises an accelerometer, a motion sensor, a proximity sensor, an optical sensor, a motion sensor, a gyrometer, a magnetometer, a proximity sensor, a hydration sensor, a force or pressure sensor, a position sensor, a global positioning sensor (GPS), an optical sensor, a magnetic sensor, a magnetometer, an inductive sensor, a capacitive sensor, an eddy current sensor, a resistive sensors, a magneto-resistive sensor, an inductive sensor, an infrared sensor, an inclinometer sensor, a piezoelectric materials or piezoelectric-based sensor, a blood-oxygen sensor, a heart-rate sensor, a laser or ultrasound based sensor, and/or an electromyography type sensor.

Some embodiments include a remote server including a computing program, applet or application configured to initiate or maintain an exchange of the usage data between the garment and the server and/or a coupled mobile computing device and the server. In some embodiments, the server is configured as a host to a web portal or coupled to a host server providing the web portal, the web portal configured to access or display the usage data or at least one parameter related to use of at least a portion of the garment. In some embodiments, the web portal is configurable to create one or more alerts directed to a user and/or a physician. In some embodiments, the alert comprises at least one of an email, a text or SMS message, a displayed icon, rendered text, a rendered graphic, a categorized or customized alert.

### BRIEF DESCRIPTION OF THE DRAWINGS

FIG. 1 shows a summary of a parent clinical study according to some embodiments.

FIG. 2 shows a summary of a rollover clinical study according to some embodiments.

FIG. 3 is a screenshot of the provider portal according to some embodiments.

FIG. 4 shows the clinical study results for knee pain, knee stiffness, and knee function when the unique therapeutic system was applied according to some embodiments.

FIG. 5 shows the clinical study results for improvements in isometric quadriceps strength according to some embodiments.

FIGS. 6 and 7 show features of the unique therapeutic system that includes a unique NMES waveform and its relationship with the unique therapeutic dose.

FIG. 8 shows a comparison of the unique waveform in comparison to conventional NMES waveforms according to some embodiments.

FIG. 9 shows a further comparison of the unique waveform in comparison to conventional NMES waveforms according to some embodiments.

FIG. 10 depicts a comparison of aspects of the unique system to various other conventional methods according to some embodiments.

FIG. 11 depicts images of the mobile app used by patients to record patient compliance according to some embodiments. In some embodiments, patients are reminded to apply their daily treatment.

FIGS. 12A-12B shows further images of the mobile app that allows both patients and physicians to track progress.

FIGS. 13A-13B illustrate a representation of a knee brace 1300 according to some embodiments.

FIGS. 14A-14B show a knee brace 1400 with one or more stabilizing bars 1410 configured to limit lateral movement of the knee according to some embodiments.

FIG. 15 illustrates various aspects of a computer system and/or controller implementing one or more aspects of the system according to some embodiments.

### DETAILED DESCRIPTION

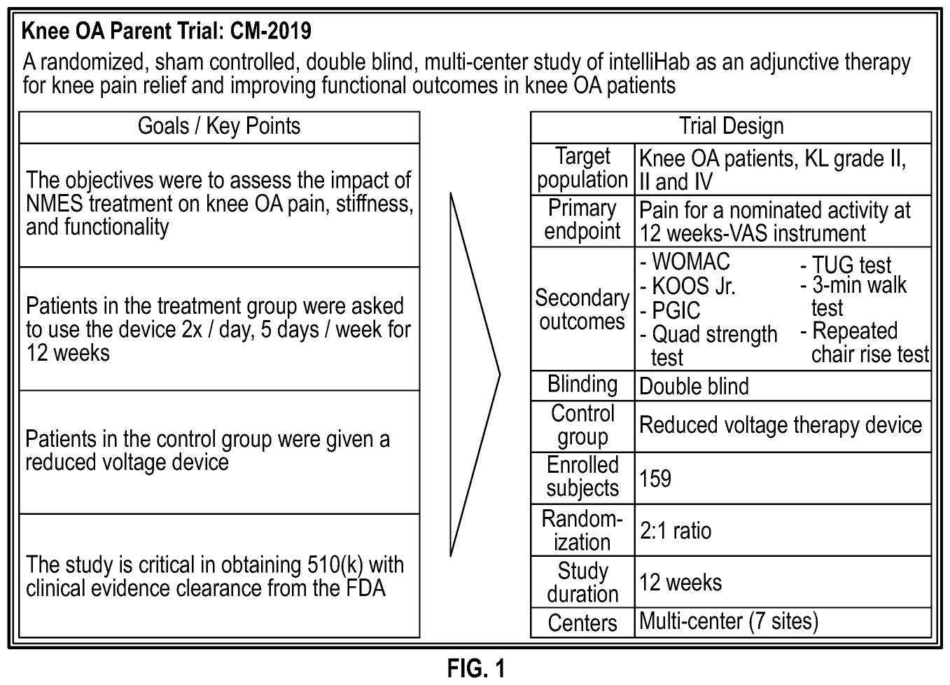

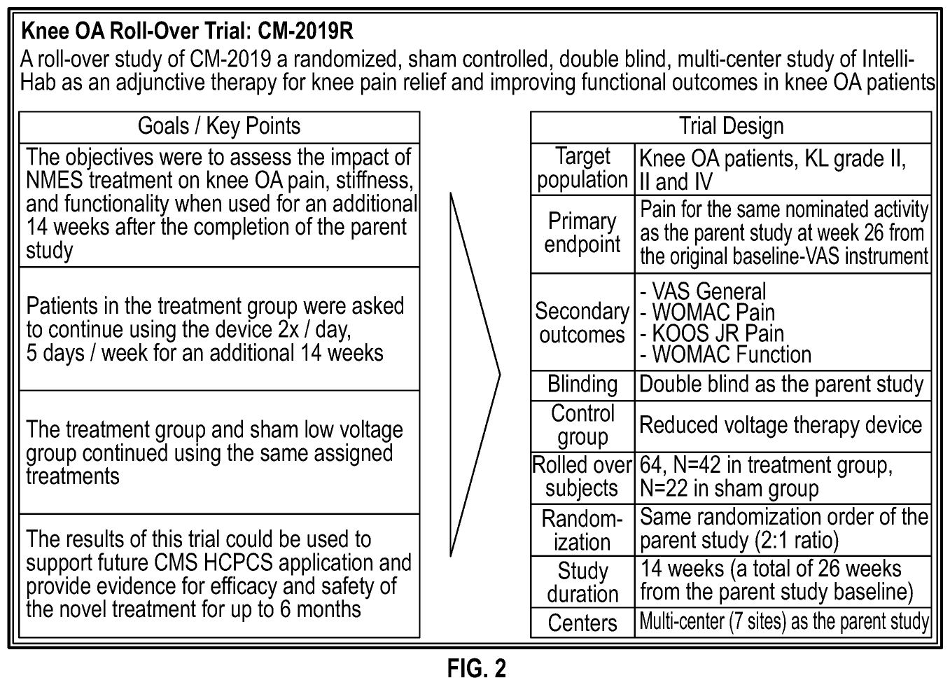

Two clinical studies (a parent study and a rollover study) were conducted on knee osteoarthritis patients using NMES therapy to evaluate the pain relief and improvements in knee joint functionality after 12 weeks of use in the parent study (summarized in FIG. 1), continued by 26 weeks of use in the rollover study (summarized in FIG. 2) in accordance with some embodiments. Two groups of patients were enrolled according to some embodiments: a treatment NMES group and a control group. The treatment NMES group received the actual CyMedica NMES treatment in accordance with some embodiments and the control group received the low voltage intensity NMES treatment. All patients were asked to apply the treatment twice a day and 5 days of the week in accordance with some embodiments. In some embodiments, each treatment session was 20 minutes which resulted in a total of 200 minutes of therapy or 110 minutes of total net muscle contraction time per week. The results of this unique therapeutic dose showed substantial improvement over prior art methods, the results of which are presented herein according to some embodiments.

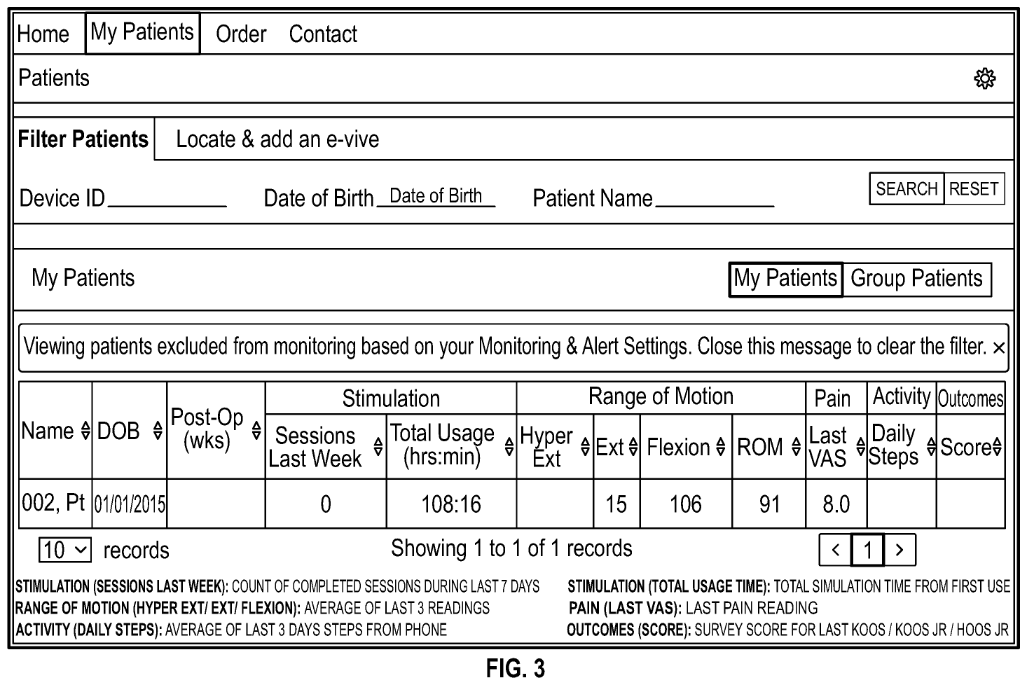

In some embodiments, patients received daily reminders on mobile apps to apply the daily treatments according to some embodiments. In some embodiments, physicians monitored compliance remotely using the provider web portal. FIG. 3 is a screenshot of the provider portal according to some embodiments.

In some embodiments, the provider portal includes a web-based provider-facing portal configured to display patient's data collected by the mobile app. In some embodiments, the database is a private SQL based database that stores the data from the patients' controller and app. In some embodiments, the database enables extraction of raw data and analytics for actual applied therapy duration compliance and applied knee and thigh intensity levels.

The duration of actual applied therapy and applied intensities according to some embodiments were measurable and reportable from each patient through the provider portal. Patients' compliance in accordance with some embodiments was monitored remotely through the course of study. The duration of actual applied therapy according to some embodiments was recorded by a mobile app, which in some embodiments was capable of transmitting the information securely to the provider portal.

The results of the two clinical studies (parent and rollover) demonstrated a unique therapeutic dose of 2 sessions a day, 5 days of the week (or 110 minutes of total net muscle contraction time per week) provided the optimum clinical benefits for the patients according to some embodiments. Some of the results of this unique dose according to some embodiments included both observed and measured significant reduction of knee pain and stiffness, as well as improvements in joint functionality, all of which were surprisingly better than suboptimal doses (any dose in these two clinical studies that were less than the unique therapeutic dose.) Additionally, some of the results of this unique therapeutic dose were surprisingly better than conventional doses. Some embodiments provided the unique therapeutic dose using a pulse waveform that initially increased substantially linearly to a peak or nearly peak level and thereafter decreased substantially exponentially without any significant level increase until the next pulse. This waveform is significantly different than the prior art or the applicant's own technology shown in its previous patents and provides a critical advantage for the efficacy of the therapeutic dose in some embodiments.

The amount of pain reduction resulting from the unique therapeutic dose according to some embodiments was also surprising and unexpected. While most of the patients in the treatment group experienced some level of pain relief, the most profound results were obtained when patients applied the prescribed dose unique therapeutic dose according to some embodiments.

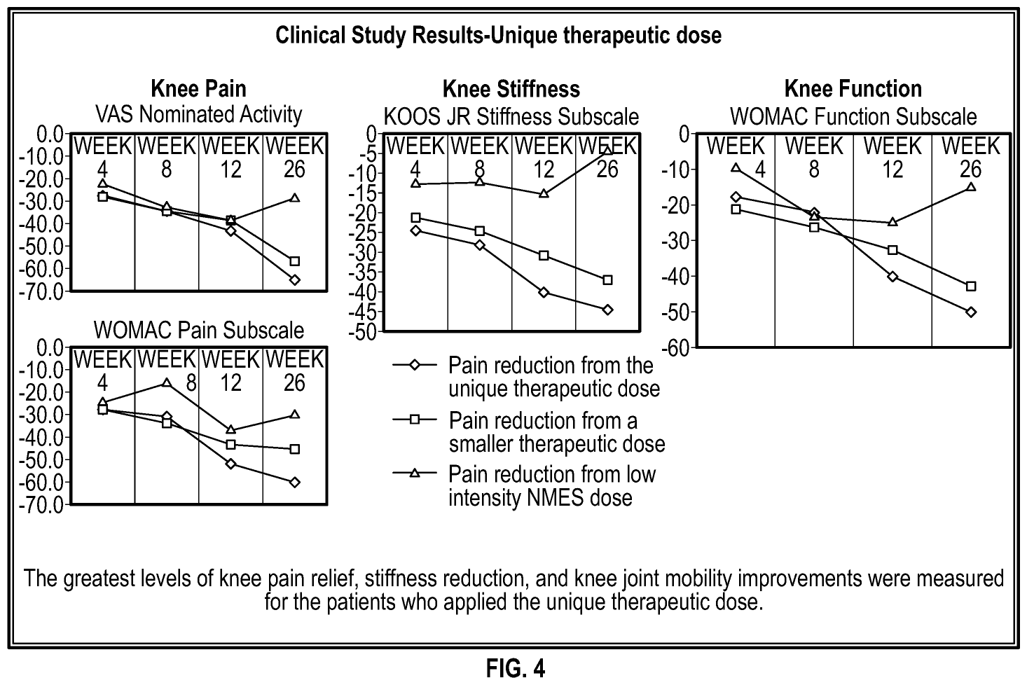

FIG. 4 shows the clinical study results for knee pain, knee stiffness, and knee function when the unique therapeutic system was applied according to some embodiments. In some embodiments, the clinical study results show the greatest levels of knee pain relief, stiffness reduction and knee joint mobility improvements measured were a result of the unique therapeutic dose.

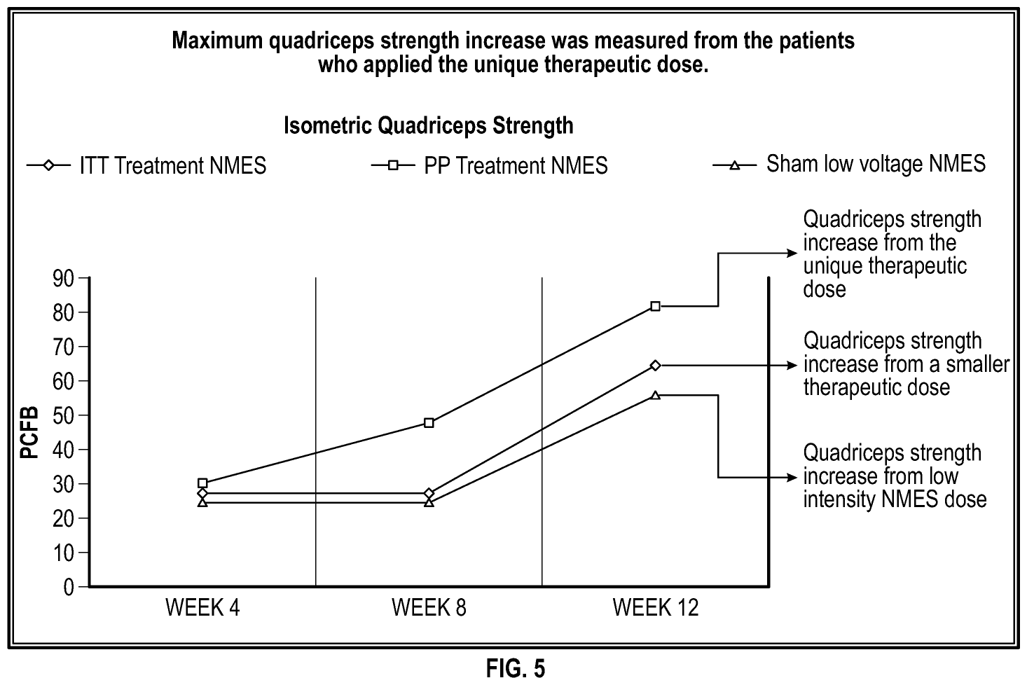

FIG. 5 shows the clinical study results for improvements in isometric quadriceps strength according to some embodiments. While a conventional low intensity NMES dose (triangle) resulted in some improvement, maximum measured quadriceps strength increase was in response to the unique therapeutic dose (square) in accordance with some embodiments.

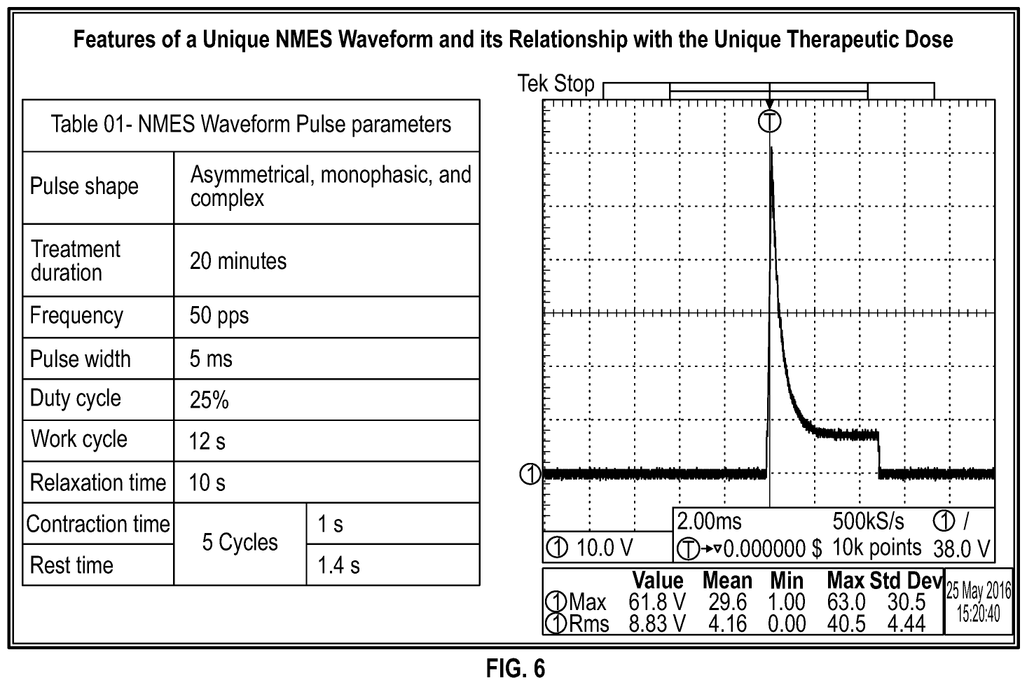

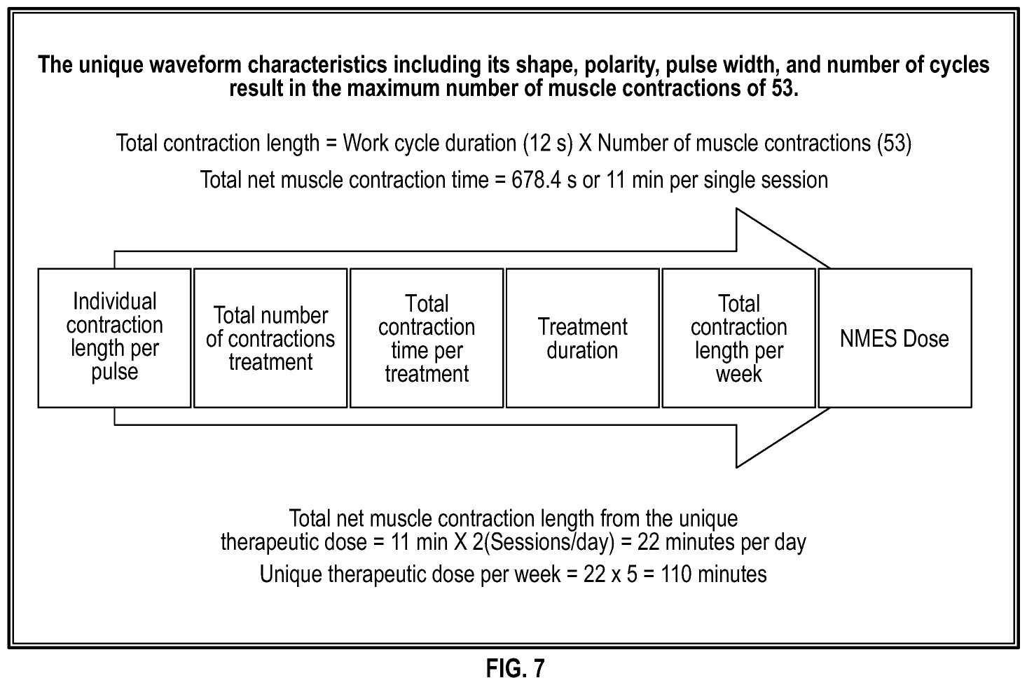

FIGS. 6 and 7 show features of the unique therapeutic system that includes a unique NMES waveform (also referred to herein as a pulse profile) and its relationship with the unique therapeutic dose. In some embodiments, the unique NMES waveform includes an asymmetrical, monophasic, and complex pulse shape. In some embodiments, the unique NMES waveform includes a treatment duration (i.e., session) of approximately 20 minutes. In some embodiments, the unique NMES waveform includes a frequency of approximately 50 pulses per second (pps). In some embodiments, the unique NMES waveform includes a duty cycle of about 25%. In some embodiments, the unique NMES waveform includes a work cycle of approximately 12 seconds. In some embodiments, the unique NMES waveform includes a relaxation time (time between pulses) of about 10 seconds. In some embodiments, the unique NMES waveform includes 5 cycles with a contraction time of 1 second and a rest time of 1.4 seconds.

In some embodiments, the unique waveform characteristics including its shape, polarity, pulse width, and number of cycles result in the maximum number of muscle contractions of 53. In some embodiments, total contraction length is determined by the following formula:

Total contraction length=Work cycle duration (12 s)×Number of muscle contractions (53)

Total net muscle contraction time according to some embodiments has been determined empirically through the clinical study and optimum results were found to be approximately 678.4 s or 11 min per single session. Total net muscle contraction length from the unique therapeutic dose on a daily basis according to some embodiments was determined to be 11 min×2(sessions/day)=22 minutes per day. The weekly time for application of the Unique therapeutic dose according to some embodiments was determined to be 22×5=110 minutes.

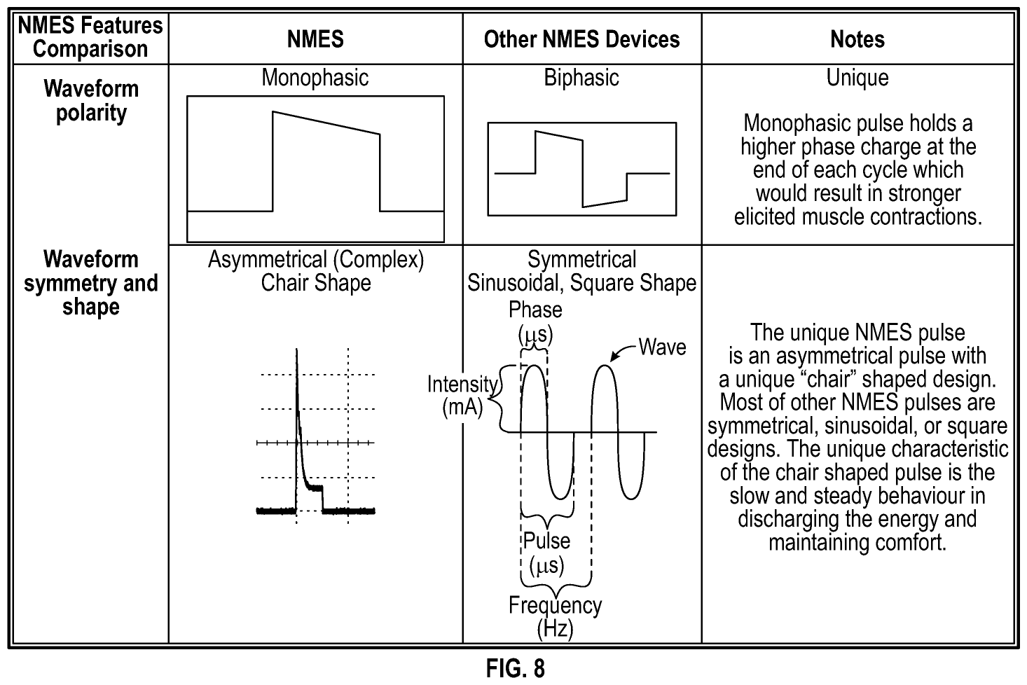

FIG. 8 shows a comparison of the unique pulse waveform in comparison to conventional NMES waveforms according to some embodiments. In some embodiments, the unique pulse according to some embodiments holds a higher phase charge at the end of each cycle which results in stronger elicited muscle contractions than the monophasic and/or biphasic conventional waveforms shown at the top of the figure. In some embodiments, the unique waveform pulse is an asymmetrical pulse with a unique “chair” shaped design. Most conventional pulses are sinusoidal or square wave in form. In some embodiments, the unique pulse includes a unique “chair shaped” electrical stimulation pulse, where the slow and steady exponential behavior in discharging the energy allows for a longer session time as it decreases patient comfort.

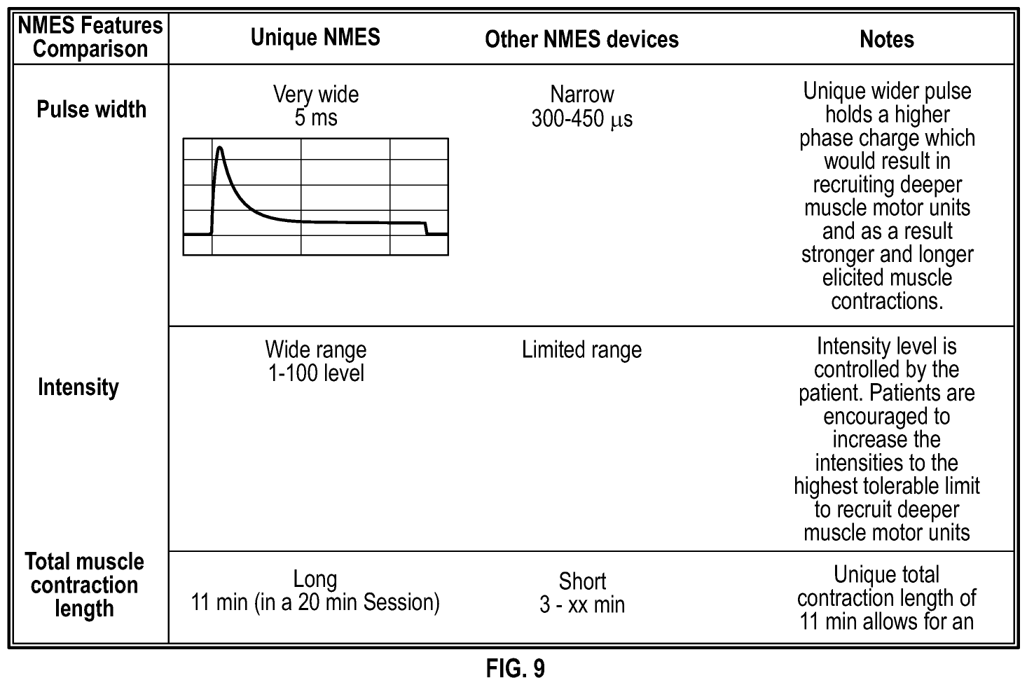

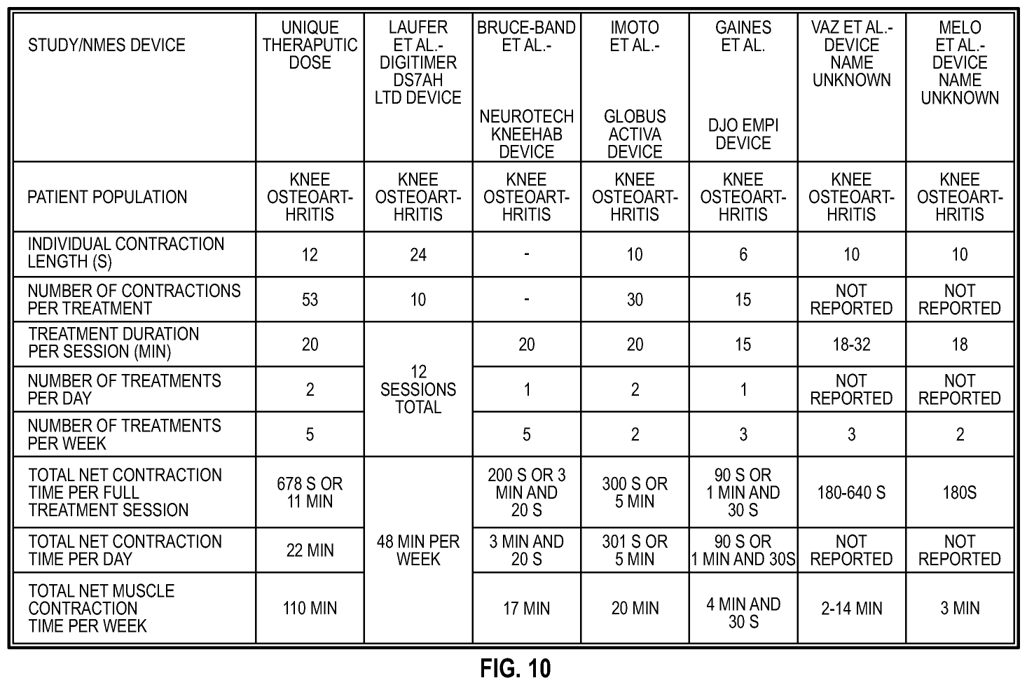

FIG. 9 shows a further comparison of the unique waveform in comparison to conventional NMES waveforms according to some embodiments. The NMES electrical stimulation pulse according to some embodiments is very wide as compared to other conventional NMES devices (5 ms vs. 300-450 μs). The unique wider pulse according to some embodiments holds a higher phase charge which results in recruiting deeper muscle motor units which results in stronger and longer elicited muscle contractions. In some embodiments, the unique NMES also includes a wider range of intensity level as compared to conventional systems (1-100 vs limited range). In some embodiments, the larger range of intensity enables patients to select an intensity that is the highest tolerable to recruit deeper muscle motor units, whereas the prior art limited range only provides for discrete jumps in intensity where between which an optimum intensity lies. In some embodiments, controller implementation of a total of 11 minutes of muscle contraction within a 20 minute therapy session program exercises the muscle much more than conventional systems that only provide a few minutes of actual muscle contraction during a 20 minute session. FIG. 10 depicts a comparison of aspects of the unique system to various other conventional methods according to some embodiments.

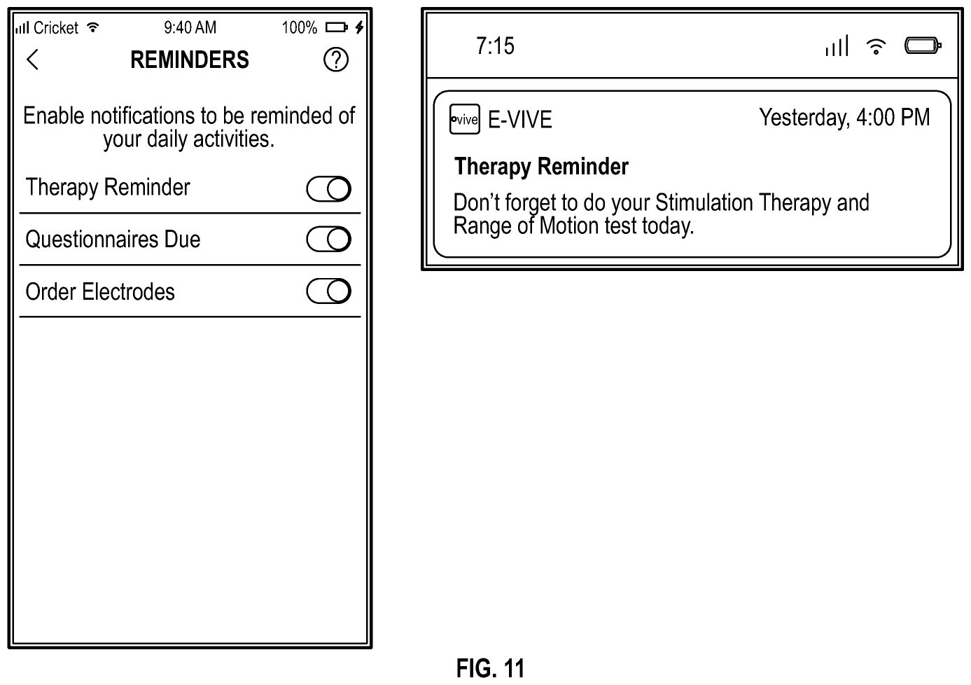

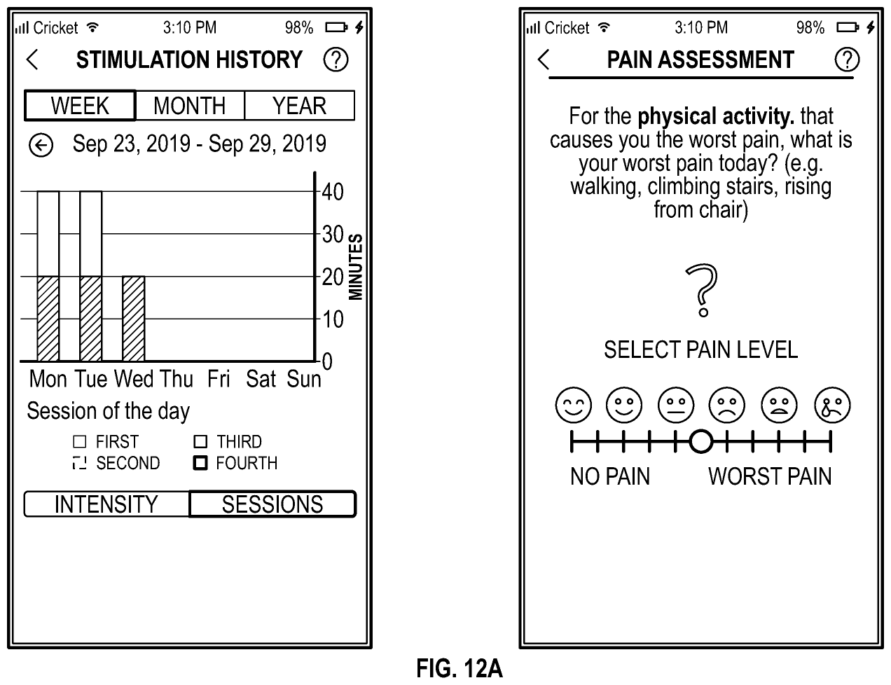

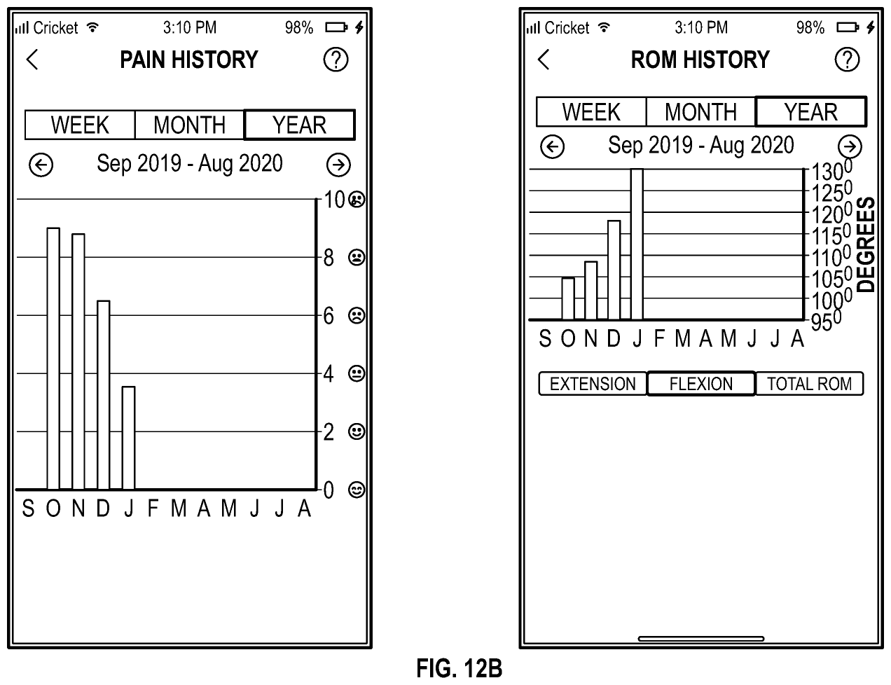

FIG. 11 depicts images of the mobile app used by patients to record patient compliance according to some embodiments. In some embodiments, patients are reminded to apply their daily treatment. FIGS. 12A-12B shows further images of the mobile app that allows both patients and physicians to track progress. In some embodiments, the mobile app includes one or more graphical user interfaces that include display and inputs for one or more of stimulation history, pain assessment, pain history, and range of motion history. In some embodiments, the mobile app and provider portal enables providers to monitor their patients' compliance remotely and to intervene when compliance is less than the recommended dose.

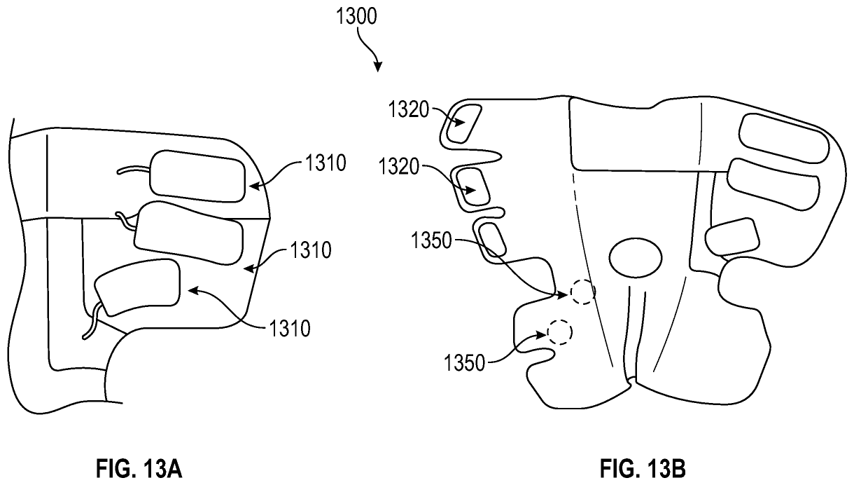

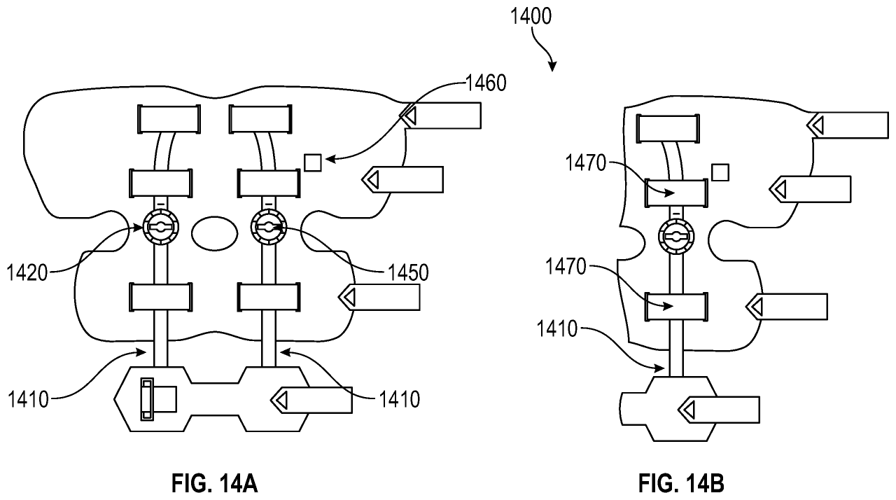

FIGS. 13A-13B illustrate a representation of a knee brace 1300 according to some embodiments. FIGS. 14A-14B show a knee brace 1400 with one or more stabilizing bars 1410 configured to limit lateral movement of the knee according to some embodiments. In some embodiments, one or more stabilizing bars 1410 are coupled together by a pivot joint 1420, where the pivot joint 1420 is configured to enable the knee brace 1400 to bend at a user's knee. In some embodiments, a knee brace 1300, 1400 comprises one or more fasteners 1330 (e.g., Velcro®, straps) configured to hold the knee brace 1300, 1400 in place when worn by a user. While a knee brace 1300, 1400 is used in this non-limiting example, any garment can be fitted with the electrodes 1310 described herein, non-limiting examples including sleeves, shorts, pants, gloves, shirts, slings, and the like. In some embodiments, one or more garments comprising aspects of the system may comprise or may not comprises one or more stabilizing bars and pivot joints.

In some embodiments, the knee brace 1300, 1400 and any of the brace systems or assemblies disclosed herein comprise one or more sensors 1350, 1450, 1460. In some embodiments, one or more sensors include one or more position sensors 1450 configured to send positional data of pivot joint 1420. In some embodiments, other sensors may include a strain gauge 1460, and/or temperature sensors 1350 as non-limiting examples. In some embodiments, one or more sensors can be integrated with and/or coupled to at least a portion of the a garment.

For example, in some embodiments, knee brace 1300, 1400 can include at least one sensor 1450 coupled to at least one of pivot joints 1420. In some embodiments, one or more portions of a wrap comprise one or more fasteners 1470 for securing one or more stabilizing bars 1410 in place. In some embodiments, the one or more garments described herein comprise a high-compression and/or non-slip material which may comprise breathable characteristics to draw out moisture and/or heat. In some embodiments, the sensors are configured to measure position, movement and/or acceleration in any x, y, and/or z-axis.

In some embodiments of the invention, any garment comprising aspects of the system described herein can include one or more controllers. In some embodiments, the controller is configured to execute one or more therapy session programs which include sensing and/or stimulation of a patient by one or more electrodes.

In some embodiments, a stimulating electrode pair implementing one or more portions of a therapy session program comprise a first electrode having a first polarity, and a second electrode having a second polarity. In some embodiments, the first and second polarities are different such that the first electrode and second electrode function to form an electrode pair capable of electrical stimulation. In some embodiments, the structure of the first electrode are substantially similar to the second electrode, while in some embodiments the first and second electrodes are different.

In some embodiments, the system comprises one or more electrical modules integrated into the garment. In some embodiments, the one or more electrical modules comprise at least one stimulation system, one or more sensor systems, and at least one display system. In some embodiments, the controller is configured to receive data from one or more modules through a wireless and/or wired connection further described herein. Further, in some embodiments, the brace system can be controlled by and/or transfer data through a controller in a wired or wireless fashion.

In some embodiments, the controller can comprise one or more computers comprising one or more processors and one or more non-transitory computer readable media. In some embodiments, the controller and or a communications module is configured to enable a medical professional to retrieve and analyze data transmitted from the system. Some embodiments of the system comprise a mobile application downloadable to one or more computer configured to enable a user and/or physician to gather data and/or control/implement one or more therapy session programs. In some embodiments, a physician and/or therapist can access data via a system web portal.

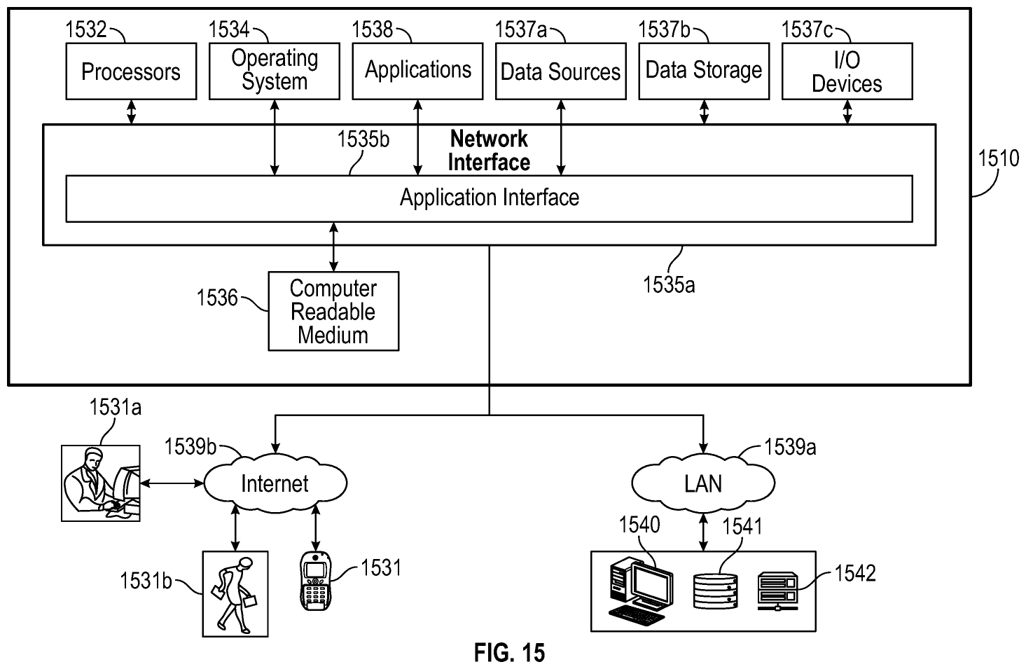

FIG. 15 illustrates a computer system 1510 enabling or comprising the systems and methods in accordance with some embodiments of the system. In some embodiments, the computer system 1510 can operate and/or process computer-executable code of one or more software modules of the aforementioned system and method. Further, in some embodiments, the computer system 1510 can operate and/or display information within one or more graphical user interfaces (e.g., HMIs) integrated with or coupled to the system.

In some embodiments, the computer system 1510 can comprise at least one processor 1532. In some embodiments, the at least one processor 1532 can reside in, or coupled to, one or more conventional server platforms (not shown). In some embodiments, the computer system 1510 can include a network interface 1535a and an application interface 1535b coupled to the least one processor 1532 capable of processing at least one operating system 1534. Further, in some embodiments, the interfaces 1535a, 1535b coupled to at least one processor 1532 can be configured to process one or more of the software modules (e.g., such as enterprise applications 1538). In some embodiments, the software application modules 1538 can include server-based software, and can operate to host at least one user account and/or at least one client account, and operate to transfer data between one or more of these accounts using the at least one processor 1532.

With the above embodiments in mind, it is understood that the system can employ various computer-implemented operations involving data stored in computer systems. Moreover, the above-described databases and models described throughout this disclosure can store analytical models and other data on computer-readable storage media within the computer system 1510 and on computer-readable storage media coupled to the computer system 1510 according to various embodiments. In addition, in some embodiments, the above-described applications of the system can be stored on computer-readable storage media within the computer system 1510 and on computer-readable storage media coupled to the computer system 1510. In some embodiments, these operations are those requiring physical manipulation of physical quantities. Usually, though not necessarily, in some embodiments these quantities take the form of one or more of electrical, electromagnetic, magnetic, optical, or magneto-optical signals capable of being stored, transferred, combined, compared and otherwise manipulated. In some embodiments, the computer system 1510 can comprise at least one computer readable medium 1536 coupled to at least one of at least one data source 1537a, at least one data storage 1537b, and/or at least one input/output 1537c. In some embodiments, the computer system 1510 can be embodied as computer readable code on a computer readable medium 1536. In some embodiments, the computer readable medium 1536 can be any data storage that can store data, which can thereafter be read by a computer (such as computer 1540). In some embodiments, the computer readable medium 1536 can be any physical or material medium that can be used to tangibly store the desired information or data or instructions and which can be accessed by a computer 1540 or processor 1532. In some embodiments, the computer readable medium 1536 can include hard drives, network attached storage (NAS), read-only memory, random-access memory, FLASH based memory, CD-ROMs, CD-Rs, CD-RWs, DVDs, magnetic tapes, other optical and non-optical data storage. In some embodiments, various other forms of computer-readable media 1536 can transmit or carry instructions to a remote computer 1540 and/or at least one user 1531, including a router, private or public network, or other transmission or channel, both wired and wireless. In some embodiments, the software application modules 1538 can be configured to send and receive data from a database (e.g., from a computer readable medium 1536 including data sources 1537a and data storage 1537b that can comprise a database), and data can be received by the software application modules 1538 from at least one other source. In some embodiments, at least one of the software application modules 1538 can be configured within the computer system 1510 to output data to at least one user 1531 via at least one graphical user interface rendered on at least one digital display.

In some embodiments, the computer readable medium 1536 can be distributed over a conventional computer network via the network interface 1535a where the system embodied by the computer readable code can be stored and executed in a distributed fashion. For example, in some embodiments, one or more components of the computer system 1510 can be coupled to send and/or receive data through a local area network (“LAN”) 1539a and/or an internet coupled network 1539b (e.g., such as a wireless internet). In some embodiments, the networks 1539a, 1539b can include wide area networks (“WAN”), direct connections (e.g., through a universal serial bus port), or other forms of computer-readable media 1536, or any combination thereof.

In some embodiments, components of the networks 1539a, 1539b can include any number of personal computers 1540 which include for example desktop computers, and/or laptop computers, or any fixed, generally non-mobile internet appliances coupled through the LAN 1539a. For example, some embodiments include one or more of personal computers 1540, databases 1541, and/or servers 1542 coupled through the LAN 1539a that can be configured for any type of user including an administrator. Some embodiments can include one or more personal computers 1540 coupled through network 1539b. In some embodiments, one or more components of the computer system 1510 can be coupled to send or receive data through an internet network (e.g., such as network 1539b). For example, some embodiments include at least one user 1531a, 1531b, is coupled wirelessly and accessing one or more software modules of the system including at least one enterprise application 1538 via an input and output (“I/O”) 1537c. In some embodiments, the computer system 1510 can enable at least one user 1531a, 1531b, to be coupled to access enterprise applications 1538 via an I/O 1537c through LAN 1539a. In some embodiments, the user 1531 can comprise a user 1531a coupled to the computer system 1510 using a desktop computer, and/or laptop computers, or any fixed, generally non-mobile internet appliances coupled through the internet 1539b. In some embodiments, the user can comprise a mobile user 1531b coupled to the computer system 1510. In some embodiments, the user 1531b can connect using any mobile computing 1531c to wireless coupled to the computer system 1510, including, but not limited to, one or more personal digital assistants, at least one cellular phone, at least one mobile phone, at least one smart phone, at least one pager, at least one digital tablets, and/or at least one fixed or mobile internet appliances.

The subject matter described herein are directed to technological improvements to the field of NMES application by providing a unique therapeutic dose to achieve significant reduction in pain and increase in muscle strength. The disclosure also describes the specifics of how a machine including one or more computers comprising one or more processors and one or more non-transitory computer implement the system and its improvements over the prior art. The instructions executed by the machine cannot be performed in the human mind or derived by a human using a pen and paper but require the machine to convert process input data to useful output data. Moreover, the claims presented herein do not attempt to tie-up a judicial exception with known conventional steps implemented by a general-purpose computer; nor do they attempt to tie-up a judicial exception by simply linking it to a technological field. Indeed, the systems and methods described herein were unknown and/or not present in the public domain at the time of filing, and they provide a technologic improvements advantages not known in the prior art. Furthermore, the system includes unconventional steps that confine the claim to a useful application.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein.

Some embodiments of the system are presented with specific values and/or setpoints. These values and setpoints are not intended to be limiting and are merely examples of a higher configuration versus a lower configuration and are intended as an aid for those of ordinary skill to make and use the system.

Furthermore, acting as Applicant's own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured.

“Simultaneously” as used herein includes lag and/or latency times associated with a conventional and/or proprietary computer, such as processors and/or networks described herein attempting to process multiple types of data at the same time. “Simultaneously” also includes the time it takes for digital signals to transfer from one physical location to another, be it over a wireless and/or wired network, and/or within processor circuitry.

As used herein, “can” or “may” or derivations there of (e.g., the system display can show X) are used for descriptive purposes only and is understood to be synonymous and/or interchangeable with “configured to” (e.g., the computer is configured to execute instructions X) when defining the metes and bounds of the system.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “container configured to receive a fluid from structure X at an upper portion and deliver fluid from a lower portion to structure Y” is limited to systems where structure X, structure Y, and the container are all disclosed as arranged to perform the recited function. The recitation “configured to” excludes elements that may be “capable of” performing the recited function simply by virtue of their construction or modification thereto but associated disclosures (or lack thereof) provide no teachings to make such a modification to meet the functional limitations between all structures recited. Another example is “a computer system configured to or programmed to execute a series of instructions X, Y, and Z.” In this example, the instructions must be present on a non-transitory computer readable medium such that the computer system is “configured to” and/or “programmed to” execute the recited instructions: “configure to” and/or “programmed to” excludes art teaching computer systems with non-transitory computer readable media merely “capable of” having the recited instructions stored thereon but have no teachings of the instructions X, Y, and Z programmed and stored thereon. The recitation “configured to” can also be interpreted as synonymous with operatively connected when used in conjunction with physical structures.

The previous detailed description is to be read with reference to the figures, in which like elements in different figures have like reference numerals. The figures, which are not necessarily to scale, depict some embodiments and are not intended to limit the scope of embodiments of the system.

Any of the operations described herein that form part of the invention are useful machine operations. The invention also relates to a device or an apparatus for performing these operations. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g. a cloud of computing resources.

The embodiments of the invention can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods can be processed by one or more machines or processors that can be connected over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data.

Although method operations are presented in a specific order according to some embodiments, the execution of those steps do not necessarily occur in the order listed unless a explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

We claim:

1. A system for providing a unique neuromuscular electrical stimulation (NMES) therapeutic dose comprising:
one or more electrodes, and
one or more controllers;
wherein the one or more controllers are configured to implement an electrical stimulation pulse is sufficient to cause muscle contraction when the one or more electrodes are placed on a user's skin;
wherein the one or more controllers are configured to execute a therapy session program;
wherein the therapy session program comprises a plurality of cycles;
wherein the plurality of cycles comprises one or more electrical stimulation time periods and one or more non-electrical relaxation time periods;
wherein the one or more electrical stimulation time periods are configured to cause the muscle contraction;
wherein the one or more non-electrical relaxation time periods are configured to not cause the muscle contraction;
wherein the one or more electrical stimulation time periods each comprise the electrical stimulation pulse which comprises a pulse profile; and
wherein the pulse profile is one or more of asymmetrical, monophasic, and complex.

2. The system of claim 1,
wherein the pulse profile comprises an initial linear increasing portion; and
wherein the pulse profile comprising an exponentially decreasing portion following a peak of the initial linear increasing portion.

3. The system of claim 2,
wherein the exponentially decreasing portion does not exponentially decrease to a substantially zero electrical power.

4. The system of claim 2,
wherein the exponentially decreasing portion does not exponentially decrease into one of the one or more non-electrical relaxation time periods.

5. The system of claim 2,
wherein when displayed on a graph, the exponentially decreasing portion comprises a substantially vertically decreasing section at a beginning;
wherein the substantially vertically decreasing section transitions to an exponentially decreasing section; and
wherein the exponentially decreasing section transitions to a substantially horizontal section at an end of the exponentially decreasing section.

6. The system of claim 5,
wherein when the graph is a graph of intensity and/or amplitude vs time, and when the pulse profile between two non-electrical relaxation time periods is defined on the graph, a scale of the graph is such that a peak on an amplitude vertical axis of the pulse profile is at least twice a width of a pulse profile time along a time horizontal axis.

7. The system of claim 5,
wherein the one or more controllers are configured to maintain a non-zero electrical intensity execution at the substantially horizontal section for a predetermined time before a zero electrical intensity execution is implemented.

8. The system of claim 7,
a final decreasing portion following the exponentially decreasing section comprises the zero electrical intensity execution by the one or more controllers.

9. The system of claim 8,
wherein the final decreasing portion comprises a substantially linear decreasing profile which joins to a non-electrical relaxation time period.

10. A system for providing a unique neuromuscular electrical stimulation (NMES) therapeutic dose comprising:
one or more electrodes, and
one or more controllers;
wherein the one or more controllers are configured to implement an electrical stimulation pulse is sufficient to cause muscle contraction when the one or more electrodes are placed on a user's skin;
wherein the one or more controllers are configured to execute a therapy session program;
wherein the therapy session program comprises a plurality of cycles;
wherein the plurality of cycles comprises one or more electrical stimulation time periods and one or more non-electrical relaxation time periods;
wherein the one or more electrical stimulation time periods are configured to cause the muscle contraction;
wherein the one or more non-electrical relaxation time periods are configured to not cause the muscle contraction;
wherein the one or more electrical stimulation time periods each comprise the electrical stimulation pulse which comprises a pulse profile; and
wherein the pulse profile is executed for a time duration between 3 and 7 milliseconds (ms).

11. The system of claim 10,
wherein a duration of the electrical stimulation pulse is between 4 and 6 ms.

12. The system of claim 10,
wherein the electrical stimulation pulse is not a square wave.

13. The system of claim 10,
wherein the electrical stimulation pulse is not sinusoidal.

14. The system of claim 10,
wherein the therapy session program is configured to execute for 17 to 23 minutes.
wherein the therapy session program comprises between 8 and 15 minutes of muscle contraction time.

15. The system of claim 11,
wherein the therapy session program is configured to execute for 19 to 21 minutes.
wherein the therapy session program comprises between 10 and 12 minutes of muscle contraction time.

16. The system of claim 10,
wherein the pulse profile comprises an initial linear increasing portion; and
wherein the pulse profile comprising an exponentially decreasing portion following a peak of the initial linear increasing portion.

17. The system of claim 16,
wherein when displayed on a graph, the exponentially decreasing portion comprises a substantially vertically decreasing section at a beginning;
wherein the substantially vertically decreasing section transitions to an exponentially decreasing section; and
wherein the exponentially decreasing section transitions to a substantially horizontal section.

18. The system of claim 17,
wherein the one or more controllers are configured to maintain a non-zero electrical intensity execution at the substantially horizontal section for a predetermined time before a zero electrical intensity execution is implemented.
